(19) United States
(12) Patent Application Publication
(10) Pub. No.: US 2006/0062814 A1
(43) Pub. Date: Mar. 23, 2006

(54) Oral compositions which mask the salty taste of salts

(76) Inventor: Roger E. Stier, Clifton, NJ (US)

Correspondence Address: KENYON & KENYON, One Broadway, New York, NY, 10004, US

(21) Appl. No.: 11/274,573

(22) Filed: Nov. 14, 2005

Related U.S. Application Data
(63) Continuation of application No. 10/418,571, filed on Apr. 17, 2003.
Application No. 10/418,571 is a continuation-in-part of application No. 10/302,092, filed on Nov. 22, 2002.

(51) Int. Cl. A61K 9/20

(52) U.S. Cl. 424/401; 424/464

(57) ABSTRACT

An oral composition or dentifrice containing a salt, such as for example, sodium bicarbonate, a zinc salt, or a strontium salt, wherein the salty taste ordinarily imparted by the salt is completely or partially masked by the combination of effective amounts of an ion-channel competitor, an initial sweetener and a delayed sweetener. The oral composition or dentifrice preferably contains sodium citrate as the ion-channel competitor, saccharin as the initial sweetener, and mono-ammonium glycyrrhizinate as the delayed sweetener.

### CROSS-REFERENCE TO RELATED APPLICATION

This application is a continuation of application Ser. No. 10/418,571, filed on Apr. 17, 2003, which is a continuation-in-part of Ser. No. 10/302,092, filed on Nov. 22, 2002, both of which are incorporated herein by reference in their entireties.

### FIELD OF THE INVENTION

The invention relates to oral compositions and dentifrices which ordinarily impart a salty taste to the mouth. More particularly, the invention relates to oral compositions and dentifrices which contain a salt, including but not limited to, sodium bicarbonate, a zinc salt, or a strontium salt, wherein the salty taste of such products is masked.

### BACKGROUND OF THE INVENTION

There are many oral compositions and dentifrices known in the art which are used to clean, refresh, preserve and/or provide treatment to the teeth and/or gums of an individual. In particular, oral compositions and dentifrices containing sodium bicarbonate (i.e., baking soda) are well known in the art of oral hygiene. Sodium bicarbonate is often added to oral compositions and dentifrices as an abrasive agent used to clean and polish the teeth.

For example, U.S. Pat. No. 4,547,362 (“Winston et al.”) discloses a sodium-bicarbonate-containing tooth powder. As described in Winston et al., “a major problem encountered in formulating a tooth powder containing sodium bicarbonate particles is the salty taste of sodium bicarbonate.” U.S. Pat. No. 4,547,362, col. 3, lines 15-17. Winston et al. attempt to address this problem of a salty taste by providing for coarser grades of sodium bicarbonate in the tooth powder disclosed therein, specifically providing for sodium bicarbonate particles having a median particle size in the range of about 74 to 210 microns. See id. at col. 3, lines 15-32; abstract. In addition, the tooth powder disclosed in Winston et al. can also include a flavoring agent, such as oils of spearmint or peppermint, and a sweetening agent, such as lactose, maltose, sorbitol, aspartame, and saccharin. According to Winston et al., the flavoring agent and sweetening agent help to mask the salty taste of the sodium bicarbonate. See id. at col. 4, lines 14-41.

U.S. Pat. No. 5,939,048 (“Alfano et al.”) discloses a pleasant-tasting oral composition which can be used to treat hypersensitive teeth. According to Alfano et al., “[d]esensitizing compositions containing strontium, potassium or other salts, not surprisingly, can have strong salty tastes . . . , including a strong salty aftertaste, [which] can reduce consumer compliance with desensitizing regimens and usage of desensitizing compositions.” U.S. Pat. No. 5,939,048, col. 1, lines 39-43. Specifically, Alfano et al. disclose a desensitizing oral composition in which sodium bicarbonate is said to mask the salty taste of a desensitizing salt, such as a potassium or strontium salt. See id. at abstract; col. 3, lines 20-40. Alfano et al. further disclose that in order to effectively mask the salty taste of the desensitizing salts in the oral composition, “sodium bicarbonate should be present from about a 1:1 ratio (on a weight basis) to the desensitizing salt to about a 6:1 ratio, although from about a 6:1 ratio to about an 8:1 ratio may also be effective.” U.S. Pat. No. 5,939,048, col. 4, lines 49-54. Although Alfano et al. disclose a formulation wherein sodium bicarbonate masks the salty taste of a desensitizing salt (e.g., potassium nitrate), it fails to teach or suggest an effective means to mask the salty taste of the sodium bicarbonate itself.

In addition, oral compositions and dentifrices containing other salts, such as for example, zinc salts and strontium salts, are well known in the art of oral hygiene, and they too pose the problem of imparting a salty taste to the user. For example, U.S. Pat. No. 5,849,266 (“Friedman”) discloses a dental composition for hypersensitive teeth. As described in Friedman, “although clinically the most effective for reducing tooth hypersensitivity, the use of strontium salts for the treatment of hypersensitivity is disliked by patients due to the tendency of strontium salts to leave an unacceptably salty taste or metallic taste in the mouth, even when used in a toothpaste form.” U.S. Pat. No. 5,849,266, col. 1, lines 53-58. Friedman attempts to address this problem by providing for a lower therapeutic level of the salt or other hypersensitivity agent over a long period of time by embedding the salt or other hypersensitivity agent “in a sustained release carrier composed of a cellulose or hydrophobic polymer, in a pharmaceutically acceptable vehicle, optionally containing a plasticizer such as polyethylene glycol and/or an adhesive polymer such as gum mastic.” Id. at col. 3, lines 12-16.

Thus, a need exists in the art for an oral composition which contains a salt, such as for example, sodium bicarbonate, a zinc salt, or a strontium salt, wherein the salty taste of the salt is effectively masked.

### SUMMARY OF THE INVENTION

It is an object of the invention to provide an oral composition or dentifrice containing a salt, wherein the salty taste ordinarily imparted by the salt is masked.

It is a further object of the invention to provide such a composition wherein the masking agents do not themselves impart a salty taste to the composition.

It is another object of the invention to provide a method for masking the salty taste of an oral composition or dentifrice containing a salt.

These and other objects of the invention which will become apparent from the following detailed description are achieved by providing an oral composition or dentifrice containing a salt, wherein the salty taste ordinarily imparted by the salt is masked by an effective amount of an ion-channel competitor, an initial sweetener and a delayed sweetener.

The ion-channel competitor is any substance which competes with the salt at the taste bud channels to diminish or lessen the firings of the salt sensors of the tongue. A preferred ion-channel competitor is sodium citrate.

In addition, the invention employs a sweetness profile which is modified to accommodate the necessity for prolonged sweetness in the presence of a high salt system. That is, the sweetness profile of an initial sweetener, such as saccharin, is initially intense with a marked decline thereafter, while the sweetness profile of a delayed sweetener, such as mono-ammonium-glycyrrhizinate, is less intense initially but builds in intensity over time. Therefore, the combination of these two sweeteners provides a sweetness profile that successfully responds to the salt-acid interaction.

It has been surprisingly discovered that the combination of these three components, an ion-channel competitor, an initial sweetener and a delayed sweetener, in an oral composition or dentifrice containing a salt, such as for example, sodium bicarbonate, a zinc salt, or a strontium salt, serves to abate or eliminate the customary salty taste attributable to the salt. The resulting oral composition or dentifrice is therefore less salty-tasting than would otherwise be expected with a salt-containing oral composition or dentifrice.

### BRIEF DESCRIPTION OF THE DRAWINGS

FIG. 1 shows taste testing results from six individual panelists comparing an ordinary toothpaste including 30% baking soda to the toothpaste of the invention according to Example 5.

FIG. 2 shows taste testing results, for the salty taste only, from six individual panelists comparing an ordinary toothpaste including 30% baking soda to the toothpaste of the invention according to Example 5.

FIG. 3 shows taste testing results from six individual panelists comparing: (1) an ordinary toothpaste including 60% baking soda; (2) the toothpaste of the invention according to Example 4; and (3) the toothpaste currently marketed as ARM & HAMMER DENTAL CARES Cool Mint Paste.

FIG. 4 shows taste testing results, for the salty taste only, from six individual panelists comparing: (1) an ordinary toothpaste including 60% baking soda; (2) the toothpaste of the invention according to Example 4; and (3) the toothpaste currently marketed as ARM & HAMMER DENTAL CARES Cool Mint Paste.

FIG. 5 shows taste testing results from six individual panelists comparing: (1) an ordinary denture adhesive composition including zinc or strontium salts (Example 6); (2) the denture adhesive composition of the invention according to Example 7; and (3) the denture adhesive composition of the invention according to Example 8.

### DETAILED DESCRIPTION OF THE INVENTION

In accordance with the invention, an oral composition or dentifrice, such as a toothpaste, containing a salt is treated with effective amounts of an ion-channel competitor, an initial sweetener and a delayed sweetener such that the salty taste typically imparted by the salt is effectively masked. That is, this combination of three components, an ion-channel competitor, an initial sweetener and a delayed sweetener, serves to abate or eliminate the customary salty taste attributable to the salt, resulting in a more pleasant-tasting oral composition or dentifrice.

Although not wishing to be bound by any theory, it is believed that the combination of these three components, an ion-channel competitor, an initial sweetener and a delayed sweetener, serves to mask the salty taste ordinarily imparted by the salt as follows.

During the experience of “tasting,” several physiological and psychological events occur simultaneously. Anatomically, taste cells reside within specialized structures called taste buds, which are located on the tongue and soft palate. The majority of taste buds are located within papillae, which are the tiny projections on the surface of the tongue that give it its velvety appearance. Taste buds are onion-shaped structures of between 50 and 100 taste cells, each of which possesses finger-like projections called microvilli that protrude through an opening at the top of the taste bud called the taste pore. Chemicals from food (called “tastants”) dissolve in saliva and contact the taste cells via the taste pore. There they either interact with surface proteins of the cells called taste receptors, as happens with sweet and bitter tastes, or they interact with pore-like proteins called ion channels, as happens with salt and sour tastes.

Physiologically and bio-chemically, the nature of the ion channel response is quite different from the surface protein (taste receptor) response. The salt taste occurs via the ion channel response, and is the response to, for example, sodium chloride (Na⁺ and Cl⁻). The sodium ions (Na⁺) enter the receptor cells via the sodium ion channels (amiloride-sensitive sodium channels). The entry of the sodium ions causes a depolarization, calcium ions (Ca⁺²) enter through voltage-sensitive calcium channels, and transmitter release occurs and results in increased firing in the primary afferent nerve. On the other hand, the sour taste is the response to acid, and acid is characterized by excess protons (H⁺). Protons block the potassium (K⁺) channels, which are responsible for maintaining the cell membrane potential at a hyperpolarized level (close to the K⁺ equilibrium potential of −85 mV). Just as described above, the blocking of these channels causes depolarization within the cell, Ca⁺² entry, transmitter release and increased firing in the primary afferent nerve. While the salt and sour taste sensations employ different channels to enter and affect the taste cells, the end result of each is very similar.

Compounds eliciting a salt or sour taste are less diverse than those eliciting a sweet taste, and they are typically ions. Generally speaking, one class of stimuli will be most effective in eliciting the highest frequency discharge. Receptor specificity is considered relative as opposed to an all-or-none response. In other words, the differences between stimuli are not so much a difference between firing and non-firing of the neurons, but is in fact the differences in the amount of firing of the neurons. This would explain why a sour compound might reduce the perception of a salty compound. That is, both respond to an ion channel mechanism which causes internal depolarization of the taste cell via a Ca⁺² mechanism leading to the neural firing. The overall taste perception of the brain will then be dependent upon the amount of firing of the receptors. By causing the receptors of sour to become engaged while the salt receptors are engaged, for example, reduces the net effect of both taste sensations to the brain.

There is also the theory that there might instead be a common receptor-neural mechanism for encoding the stimuli that produce a taste response, with separate mechanisms for coding salty, sour, and sweet tastes. As taste typically occurs in dynamic conditions of stimulation involving interactions between various substances, numerous complex interactions among the primary taste qualities prevent the predicting of the precise product of taste mixtures. The result of combining two chemical solutions whose single components each evoke a different taste is a complex psychophysiological event: the solutions do not function independently of each other, but, depending upon the chemical substances, may show facilitative or inhibitory effects in combination. A mixture of different tastes will only elicit responses to those two tastes and will not produce tastes not present. For example, a salt taste combined with a bitter taste will only produce a salt taste and a bitter taste—it will not produce a sweet and/or sour taste. There is evidence then that taste qualities may mutually suppress each other. In the case of a strong salt taste with a weaker acidic taste the salt taste would become diminished. That is, while retaining their taste qualities within the mixture, they are perceived as being less intense than when they are in pure, unmixed solutions. However, even if two sapid substances of the taste mixture are exceedingly strong, they cannot mutually suppress each other to produce a tasteless mixture.

Again, although not wishing to be bound by any particular theory, it is believed that the foregoing discussion explains how the combination of the three masking components of the invention (an ion-channel competitor, an initial sweetener and a delayed sweetener) effectively masks the customary salty taste attributable to a salt. That is, from a physiological standpoint, the perception of the salt taste and the sour taste employ the same receptor mechanism of ion channel transfer. Although they use different channels to enter the taste cell, the mechanism they use for firing the neuron once inside the taste cell is identical. It is no longer believed that there exists a “tongue map” for taste buds with each area of the tongue perceiving only certain sensations; instead, it is believed that taste cells respond to all sensations in different ways. The brain understands the experience of “taste” not as fired neurons or non-fired neurons, but as the amount of firing of the neurons. If a salt is present in sufficient concentration the neurons will be firing rapidly from the salt channels of all cells. However, the introduction of a sour note in addition to the salt complicates the overall neuron response, as neural responses to each taste (salt and sour) produce responses in competition with each other. Thus, the strong taste of a 60% salt composition (such as sodium bicarbonate) now becomes less intense in the presence of an acid or an acidic salt.

The salt of the oral composition or dentifrice of the invention is any salt which ordinarily imparts a salty taste to the oral composition or dentifrice. For example, these salts include, but are not limited to: metallic salts of the chlorines, fluorines, phosphates, and carbonates; zinc salts; strontium salts; stannous salts; and combinations thereof. Examples of such salts include, but are not limited to, sodium bicarbonate, zinc chloride, zinc citrate, zinc acetate, zinc gluconate, zinc lactate, zinc salicylate, zinc sulphate, zinc fluoride, potassium chloride, potassium bicarbonate, tetrasodium pyrophosphate, sodium polyphosphate, strontium chloride, and stannous fluoride. In addition, as would be understood by one of ordinary skill in the art, the amount of the salt or salts present in the oral composition or dentifrice of the invention will vary depending upon the particular salt or salts employed in the oral composition or dentifrice, and all such amounts of the salt or salts are within the scope of the present invention.

The ion-channel competitor which is used in the oral composition or dentifrice of the invention is any substance that competes with the salt at the appropriate channels within the taste buds to diminish or lessen the firings of the salt sensors. Examples of suitable ion-channel competitors include, but are not limited to: sodium salts of citric acid (sodium citrates); calcium salts of citric acid (calcium citrates); sodium salts of phosphoric acid (sodium phosphates); monobasic calcium salts of phosphoric acid; and salts of hydroxy acids, which include glycolic, lactic, hydroxybutyric, mandeliec, glycergic, malic, tartaric, and mesotartaric acids (such salts including sodium and calcium as well as for tartaric acid, dipotassium, dissodium, and diammonium). Preferably, the ion-channel competitor is or includes sodium citrate.

The amount of ion-channel competitor to be included in the oral composition or dentifrice of the invention is any amount which is effective to mask the customary salty taste attributable to the salt, when combined with effective amounts of the initial sweetener and the delayed sweetener. For example, when the ion-channel competitor comprises sodium citrate, the amount of sodium citrate to be included in the oral composition or dentifrice is preferably from about 0.25% to about 2.0% of the total weight of the oral composition or dentifrice, and most preferably about 1.0% of the total weight of the oral composition or dentifrice.

The oral composition or dentifrice of the invention also includes an initial sweetener that provides an initial, intense sweetness to help abate or eliminate the customary salty taste of the salt. Examples of the initial sweetener include, but are not limited to: saccharin; sucralose; neotame; alitame; aspartame; cyclamate; thaumatin; dihydrochalcones; and acesulfame potassium (acesulfame K) compounds. Preferably, the initial sweetener is or includes saccharin. The intensity of the sweetness which is rapidly imparted by the initial sweetener declines rapidly with time.

The amount of initial sweetener to be included in the oral composition or dentifrice of the invention is any amount which is effective to mask the customary salty taste attributable to the salt, when combined with effective amounts of the ion-channel competitor and the delayed sweetener. For example, when the initial sweetener comprises saccharin, the amount of saccharin to be included in the oral composition or dentifrice is preferably from about 0.10% to about 0.80% of the total weight of the oral composition or dentifrice, and most preferably about 0.60% of the total weight of the oral composition or dentifrice.

In addition, the oral composition or dentifrice of the invention includes a delayed sweetener that provides a less intense sweetness initially which builds in intensity over time to extend the sweetness profile and help abate or eliminate the customary salty taste of the salt. Preferably, the delayed sweetener is or includes mono-ammonium glycyrrhizinate (“MAG”).

The amount of delayed sweetener to be included in the oral composition or dentifrice of the invention is any amount which is effective to mask the customary salty taste attributable to the salt, when combined with effective amounts of the ion-channel competitor and the initial sweetener. For example, when the delayed sweetener comprises mono-ammonium glycyrrhizinate, the amount of mono-ammonium glycyrrhizinate to be included in the oral composition or dentifrice is preferably from about 0.05% to about 0.50% of the total weight of the oral composition or dentifrice, and most preferably about 0.30% of the total weight of the oral composition or dentifrice.

The oral composition or dentifrice of the invention may be in any of the forms known in the art, including but not limited to, a toothpaste, a mouthwash, a tooth powder, a chewing gum, a dental cream or gel, and a denture adhesive composition. Preferably, the oral composition or dentifrice of the invention is in the form of a toothpaste.

In general, the oral compositions or dentifrices of this invention are prepared utilizing techniques well known to those of ordinary skill in the art. As such, the oral compositions or dentifrices of this invention may include various other components which are customarily used in the preparation of such oral compositions and dentifrices, such as, for example, toothpastes and dental creams and gels.

When the oral composition of the invention is in the form of a toothpaste, or a dental cream or gel, such a form typically includes a liquid carrier material for the active ingredients which mask the salty taste of the salt. The carrier material may comprise water, typically in an amount of from about 10% to about 90% by weight of the oral composition. Carrier materials include, but are not limited to, polyethylene glycol (PEG), propylene glycol, glycerin or mixtures thereof. In addition, the oral composition may include humectants, such as, for example, sorbitol, glycerin, and polyalcohols. Particularly advantageous liquid ingredients comprise mixtures of water with polyethylene glycol or glycerin and sorbitol. A gelling agent (thickening agent) including natural or synthetic gums, such as sodium carboxymethylcellulose, hydroxyethyl cellulose, methyl cellulose and the like, may also be used, typically in the range of about 0.5% to about 5% by weight of the oral composition. In a toothpaste, dental cream or gel, the liquids and solids are proportioned to form a creamy or gelled mass which is extrudable from a pressurized container or from a collapsible tube.

The toothpaste or dental cream or gel of the invention may also contain a surface active agent (i.e., surfactant) which may be an anionic, nonionic or zwitterionic detergent, typically present in amounts of from about 0.05% to about 5% by weight of the oral composition. Suitable anionic and nonionic surfactants are well known in the art. For example, suitable anionic surfactants include, but are not limited to, anionic high-foam surfactants, such as linear sodium C₁₂₋₁₈ alkyl sulfates; sodium salts of C₁₂₋₁₆ linear alkyl polyglycol ether sulfates containing from 2 to 6 glycol ether groups in the molecule; alkyl-(C₁₂₋₁₆)-benzene sulfonates; linear alkane-(C₁₂₋₁₈)-sulfonates; sulfosuccinic acid mono-alkyl-(C₁₂₋₁₈)-esters; sulfated fatty acid monoglycerides; sulfated fatty acid alkanolamides; sulfoacetic acid alkyl-(C₁₂₋₁₈)-esters; and acyl sarcosides, acyl taurides and acyl isothionates all containing from 8 to 18 carbon atoms in the acyl moiety. In addition, examples of suitable nonionic surfactants include, but are not limited to, ethoxylates of fatty acid mono- and diglycerides, fatty acid sorbitan esters and ethylene oxide-propylene oxide block polymers. Particularly preferred surfactants are sodium lauryl sulfate and sacrosinate, and combinations of surfactants can also be used.

Zwitterionic surface active agents include the betaines and sulfobetaines. Typical alkyl dimethyl betaines include decyl betaine or 2-(N-decyl-N,N-dimethylammonio) acetate, coco betaine, myristyl betaine, palmityl betaine, lauryl betaine, cetyl betaine, stearyl betaine, etc. The amidobetaines similarly include cocoamidoethyl betaines, cocoamidopropyl betaine, lauramidopropyl betaine and the like. These sulfobetaines are similar in structure to the betaines, but have a sulfonate group in place of the carboxylate group, and include alkylsulfobetaines, alkylamidosulfobetaines and alkylaminosulfobetaines.

Any of the customary abrasives or polishes may also be used in the invention, including those selected from the group consisting of chalk, calcium carbonate, dicalcium phosphate, insoluble sodium metaphosphate, aluminum silicates, calcium pyrophosphate, finely particulate synthetic resins, silicas, aluminum oxide, aluminum oxide trihydrate, hydroxyapatite, and the like, or combinations thereof. The abrasive or polishes may, preferably, be completely or predominantly finely particulate xerogel silica, hydrogel silica, precipitated silica, aluminum oxide trihydrate and finely particulate aluminum oxide or combinations thereof. Silicas available from J.H. Huber Corporation, Havre de Grace, Md., U.S.A. under the trade names ZEOFREE® and ZEODENT® may be used in the invention.

Preservatives and antimicrobial agents that may be used in the toothpaste or gels of the invention include those selected from the group consisting of p-hydroxybenzoic acid, methyl, ethyl or propyl ester, sodium sorbate, sodium benzoate, bromochlorophene, phenyl salicylic acid esters, thymol, and the like, and combinations thereof. Suitable pH buffers include those selected from the group consisting of primary, secondary or tertiary alkali phosphates, citric acid, sodium citrate, and the like or combinations thereof. Wound healing and inflammation inhibiting substances include those selected from the group consisting of allantoin, urea, azulene, camomile active substances and acetyl salicylic acid derivatives, and the like, or combinations thereof.

The oral composition or dentifrice of this invention may also include a thickening agent or binder. For example, the thickening agent or binder may be selected from the group consisting of finely particulate gel silicas and nonionic hydrocolloids, such as carboxymethyl cellulose, sodium hydroxymethyl cellulose, hydroxyethyl cellulose, hydroxypropyl guar, hydroxyethyl starch, polyvinyl pyrrolidone, vegetable gums, such as tragacanth, agar agar, carrageenans, gum arabic, xanthan gum, guar gum, locust bean gum, carboxyvinyl polymers, fumed silica, silica clays and the like, and combinations thereof. A preferred thickening agent is carrageenan available under the trade names GELCARIN® and VISCARIN® from FMC Biopolymers, Philadelphia, Pa., U.S.A. Other thickening agents or binders are polyvinyl pyrrolidone available from Noveon, Inc. Cleveland, Ohio, U.S.A. under the trademark CARBOPOL®, fumed silica under the trademark CAB-O-SIL® available from Cabot Corporation, Boston, Mass., U.S.A., and silica clays available from Laporte Industries, Ltd., London, U.K. under the trademark LAPOINTE®. The thickening agent or binder may be used with or without a carrier, such as glycerol, polyethylene glycol (e.g., PEG-400), or combinations thereof, however, when a carrier is used, up to about 5% thickening agent or binder, preferably from about 0.1% to about 1.0%, is combined with about 95.0% to about 99.9% carrier, preferably about 99.0% to about 99.9%, based on the total weight of the thickening agent/carrier combination.

Active compounds may also be present in the oral composition or dentifrice of the invention, depending upon the nature and use of the oral composition or dentifrice. In general, the active compounds for oral compositions mask oral malodor, attack the chemicals that bring about the oral malodor, kill or inhibit growth of the bacteria in the mouth that cause breath malodor or halitosis, attack tartar, remove dirt from the teeth and mouth and/or whiten teeth. For example, in embodiments of the invention where the oral compositions are in the form of mouthwashes, mouth rinses, gums, mouth sprays, lozenges and the like, the active components may include oral hygiene actives, antibacterial substances, desensitizing agents, antiplaque agents and combinations thereof, such as those, for example, selected from the group consisting of chlorine dioxide, fluoride, alcohols, triclosan, domiphen bromide, cetyl pyridinium chlorine, calcium lactate, calcium lactate salts and the like, and combinations thereof. As a further example, in embodiments of the invention where the oral compositions are in the form of dentifrices, such as toothpastes, gels, and the like, the active components may include oral hygiene actives, antibacterial substances, desensitizing agents, antiplaque agents and combinations thereof, such as those, for example, selected from the group consisting of sodium fluoride, stannous fluoride, sodium monofluorophosphate, triclosan, cetyl pyridium chloride, zinc salts, pyrophosphate, calcium lactate, calcium lactate salts, 1-hydroxyethane-1,2-diphosphonic acid, 1-phosphonopropane-1,2,3-tricarboxylic acid, azacycloalkane-2,2-diphosphonic acids, cyclic aminophosphonic acids and the like, and combinations thereof.

In addition, the oral composition or dentifrice of this invention may also include a sugar alcohol and/or a flavor agent to enhance the overall taste of the oral composition or dentifrice. The sugar alcohols that may be employed in the invention are any of those known in the art which have effective sweetening capabilities. Generally, the sugar alcohols are selected from the group consisting of sorbitol, xylitol, mannitol, maltitol, hydrogenated starch hydrolysate, and mixtures thereof, with sorbitol being the preferred sugar alcohol. The flavor agent or agents that may be used in the invention include those known to the skilled artisan, such as, natural and artificial flavors. These flavor agents may be chosen from synthetic flavor oils and flavoring aromatics, and/or oils, oleo resins and extracts derived from plants, leaves, flowers, fruits and so forth, and combinations thereof. Representative flavor oils include: cinnamon oil, peppermint oil, clove oil, bay oil, eucalyptus oil, thyme oil, cedar leaf oil, oil of nutmeg, oil of sage, and oil of bitter almonds. Also useful are artificial, natural or synthetic fruit flavors such as vanilla, and citrus oil, including lemon, orange, grape, lime and grapefruit and fruit essences including apple, pear, peach, strawberry, raspberry, cherry, plum, pineapple, apricot and so forth. Any of these flavor agents may be used individually or in admixture. Commonly used flavors include mints such as peppermint, menthol, artificial vanilla, cinnamon derivatives, and various fruit flavors, whether employed individually or in admixture. Flavor agents such as aldehydes and esters including cinnamyl acetate, cinnamaldehyde, citral, diethyllacetal, dihydrocarvyl acetate, eugenyl formate, p-methylanisole, and so forth may also be used. Generally, any flavoring or food additive such as those described in Chemicals Used in Food Processing, pub 1274 by the National Academy of Sciences, pages 63-258 may be used as flavor agents in the invention.

The oral composition or dentifrice of this invention may also contain coloring agents or colorants, such as colors, dyes, pigments and particulate substances, in amounts effective to produce the desired color. The coloring agents (colorants) useful in the invention include the pigments such as titanium dioxide, which may be incorporated in amounts of up to about 2% by weight of the oral composition, and preferably less than about 1% by weight. Colorants may also include natural food colors and dyes suitable for food, drug and cosmetic applications. For example, food grade and/or pharmaceutically acceptable coloring agents, dyes, or colorants, as would be understood to one skilled in the art, include FD&C colorants such as primary FD&C Blue No. 1, FD&C Blue No. 2, FD&C Green No. 3, FD&C Yellow No. 5, FD&C Yellow No. 6, FD&C Red No. 3, FD&C Red No. 33 and FD&C Red No. 40 and lakes FD&C Blue No. 1, FD&C Blue No. 2, FD&C Yellow No. 5, FD&C Yellow No. 6, FD&C Red No. 2, FD&C Red No. 3, FD&C Red No. 33, FD&C Red No. 40 and combinations thereof.

### EXAMPLES

Specific preferred embodiments of the invention will now be described with reference to the following examples which should be regarded in an illustrative rather than a restrictive sense.

The flavor agent used in any of the following Examples included the following components:

| Flavor Agent Component | percent by weight |
|---|---|
| Anethole USP | 10% |
| Menthol Crystals USP (large) N | 18.75% |
| Menthol Synthetic | 6.25% |
| Menthone Laevo | 3.0% |
| Menthyl Acetate R.F. | 1.0% |
| Peppermint NF (lot #0058-9) N | 3.0% |
| Peppermint Red Farwest #0507-9 N | 10% |
| Peppermint Red Idaho N (Leman) | 3.0% |
| Peppermint Red Madras N | 10% |
| Peppermint Red Midwest N (Leman) | 35% |

The mono-ammonium glycyrrhizinate used in any of the following Examples was MAGNASWEET® 120 (Mafco). MAGNASWEET® 100, 125, 130, 165 and 365 can also be used in accordance with the invention.

### Examples 1-3

The toothpastes of Examples 1-3 were prepared using techniques well-known to those of ordinary skill in the art. Specifically, the toothpastes of Examples 1-3 were prepared as follows. A first phase was prepared by dispersing the carboxymethyl cellulose (CMC 7M8SXF) in the polyethylene glycol. A second phase was then prepared by combing 34.0 grams of water and 101.5 grams of sorbitol, then dissolving therein the sodium citrate, saccharin, sodium fluoride, and mono-ammonium glycyrrhizinate, and heating to 50 degrees Celsius. The first phase was then added to the second phase and mixed for 20 minutes under high shear. This mixture was then transferred to a Ross mixer. Next, a third phase was prepared by combining the sodium bicarbonate and titanium dioxide. This third phase is then added to the Ross mixer over a 15-minute period of time. Once this addition is completed, the vacuum is raised to 28″ and mixed for 15 minutes. The formula amount of the flavor agent is then added to the mixer and mixed for 10 minutes at 28″ vacuum. Next, a fifth phase was prepared by dissolving the sodium lauryl sulfate in 25 grams of water. The Ross mixer is stopped and the vacuum reduced to atmospheric pressure. The fifth phase is then added to the mixer and vacuum is raised to 28″. The Ross mixer is then started and mixed for 10 minutes. Upon completion of this final mixing, the mixer is stopped, the vacuum is released, and the batch is transferred to appropriate storage vessels for future use.

The toothpastes prepared according to Examples 1-3 have the following ingredients:

| | Example 1 | | Example 2 | | Example 3 | |
|---|---|---|---|---|---|---|
| | wt. % | | wt. % | | wt. % | |
| | of oral | weight | of oral | weight | of oral | weight |
| Ingredient | comp. | (grams) | comp. | (grams) | comp. | (grams) |
| PEG 400 | 3 | 15 | 3 | 15 | 3 | 15 |
| CMC 7M8SXF | 0.6 | 3 | 0.75 | 3.75 | 0.6 | 3 |
| H₂0 | 6.8 | 34 | 6.8 | 34 | 6.8 | 34 |
| sorbitol | 20.3 | 101.5 | 20.85 | 104.75 | 19.85 | 99.25 |
| sodium citrate | 1 | 5 | 1 | 5 | 1 | 5 |
| sodium fluoride | 0.2 | 1 | 0.2 | 1 | 0.2 | 1 |
| saccharin | 0.5 | 2.5 | 0.8 | 4 | 0.6 | 3 |
| mono- | 0.2 | 1 | 0.2 | 1 | 0.3 | 1.5 |
| ammonium | | | | | | |
| glycyrrhizinate | | | | | | |
| sodium | 60 | 300 | 60 | 300 | 60 | 300 |
| bicarbonate | | | | | | |
| TiO₂ | 0.25 | 1.25 | 0.25 | 1.25 | 0.25 | 1.25 |
| flavor agent | 1 | 5 | — | — | 1.25 | 6.25 |
| H₂0 | 5 | 25 | 5 | 25 | 5 | 25 |
| sodium lauryl | 1.15 | 5.75 | 1.15 | 5.75 | 1.15 | 5.75 |
| sulfate | | | | | | |

### Examples 4-5

The toothpastes of Examples 4-5 were prepared using techniques well-known to those of ordinary skill in the art. Specifically, the toothpaste of Example 4 was prepared according to the same procedure as employed in Examples 1-3 above.

The toothpaste of Example 5 was prepared as follows. A first phase was prepared by dispersing the carboxymethyl cellulose (CMC 12M 31 XP) and the carrageenan (Gelcarin DG) in the polyethylene glycol. A second phase was then prepared by combining 50 grams of water with 205.25 grams of sorbitol, then dissolving therein the saccharin, sodium fluoride, sodium citrate, and the mono-ammonium glycyrrhizinate, and heating to 60 degrees Celsius. The first phase is then added to the second phase and mixed for 20 minutes under high shear. This mixture is then transferred to a Ross mixer. A third phase is then formed by combing the sodium bicarbonate, the titanium dioxide, and the silica (Huber Zeofree 153). This third phase is then added to the Ross mixer over 15 minutes at atmospheric pressure. Once this addition is completed, the vacuum is raised to 28″ and mixing continues for another 15 minutes. The formula amount of the flavor agent is then added to the mixer and mixed for 10 minutes at 28″ vacuum. Next, a fifth phase is prepared by dissolving the sodium lauryl sulfate in 25 grams of water. The Ross mixer is then stopped, and the vacuum is released. The fifth phase is then added to the mixer, the vacuum is increased to 28″, and the batch mixes for 10 minutes. Once these 10 minutes of mixing are completed, the mixer is stopped and the vacuum released. The finished toothpaste is then transferred to appropriate containers for future use.

The toothpastes prepared according to Examples 4-5 have the following ingredients:

| | Example 4 | | Example 5 | |
|---|---|---|---|---|
| | wt. % of oral | weight | wt. % of oral | weight |
| Ingredient | composition | (grams) | composition | (grams) |
| PEG 400 | 3 | 15 | 3 | 15 |
| CMC 7M8SXF | 0.6 | 3 | — | — |
| CMC 12M31XP | — | — | 1 | 5 |
| carrageenan (gelcarin | — | — | 0.25 | 1.25 |
| DG) | | | | |
| H₂0 | 6.8 | 34 | 10 | 50 |
| sorbitol | 19.9 | 99.5 | 41.05 | 205.25 |
| sodium citrate | 1 | 5 | 1 | 5 |
| sodium fluoride | 0.2 | 1 | 0.2 | 1 |
| saccharin | 0.6 | 3 | 0.6 | 3 |
| mono-ammonium | 0.3 | 1.5 | 0.3 | 1.5 |
| glycyrrhizinate | | | | |
| sodium bicarbonate | 60 | 300 | 30 | 150 |
| TiO₂ | 0.25 | 1.25 | 0.25 | 1.25 |
| silica (zeofree 153) | — | — | 5 | 25 |
| H₂0 | 5 | 25 | 5 | 25 |
| sodium lauryl sulfate | 1.15 | 5.75 | 1.15 | 5.75 |
| flavor agent | 1.2 | 6 | 1.2 | 6 |

Taste testing of the toothpastes prepared according to Examples 4 and 5 above was conducted by a six-member panel.

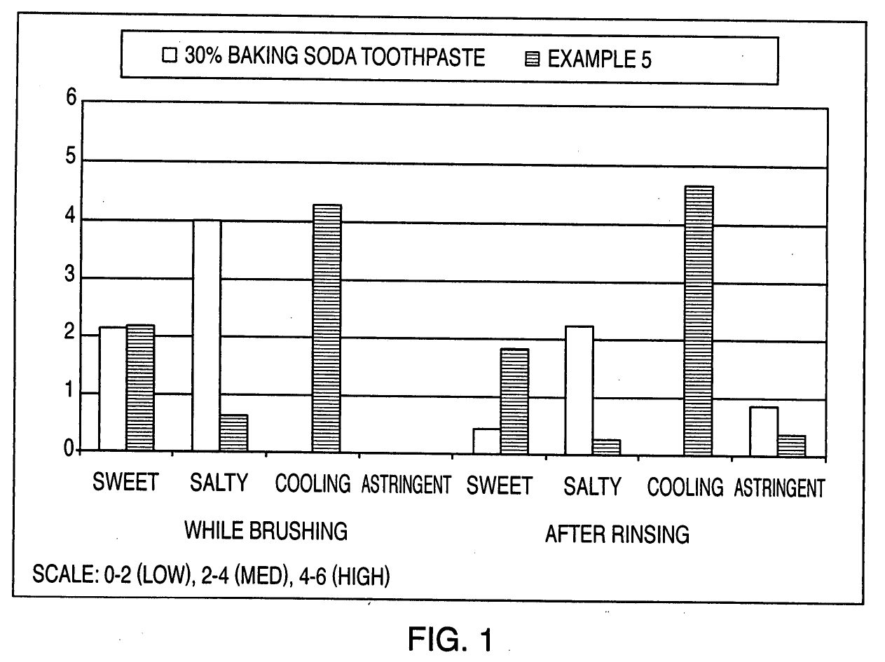

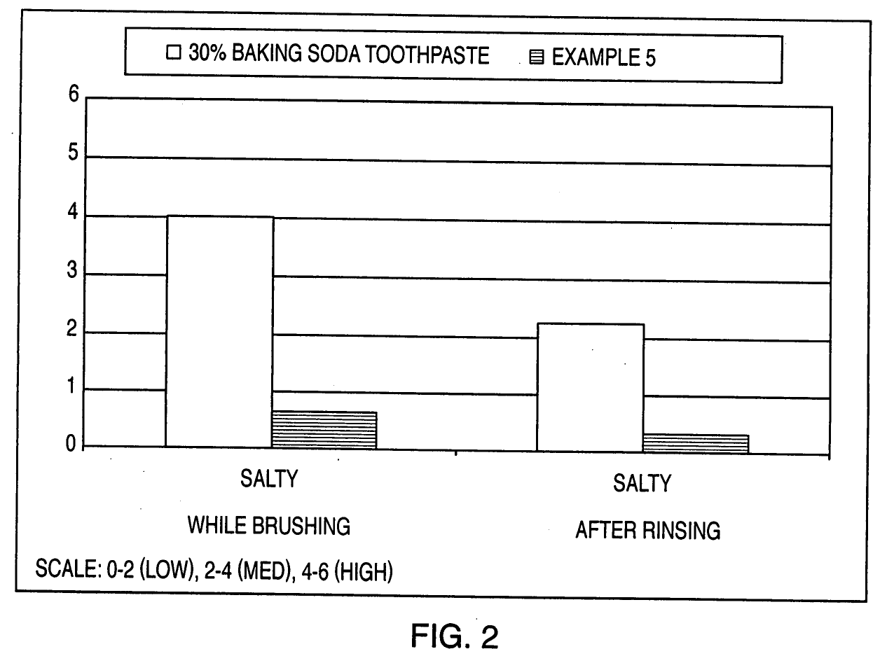

For example, FIGS. 1 and 2 show taste testing results from six individual panelists comparing an ordinary toothpaste including 30% baking soda to the toothpaste of the invention according to Example 5. As can be seen in FIGS. 1 and 2, the salty taste ordinarily imparted by the 30% baking soda is greatly diminished, and almost entirely removed, in the toothpaste of the invention according to Example 5, both while brushing and after rinsing. In addition, FIG. 1 shows that the toothpaste of the invention according to Example 5 imparts a cooling taste not present in the ordinary toothpaste including 30% baking soda.

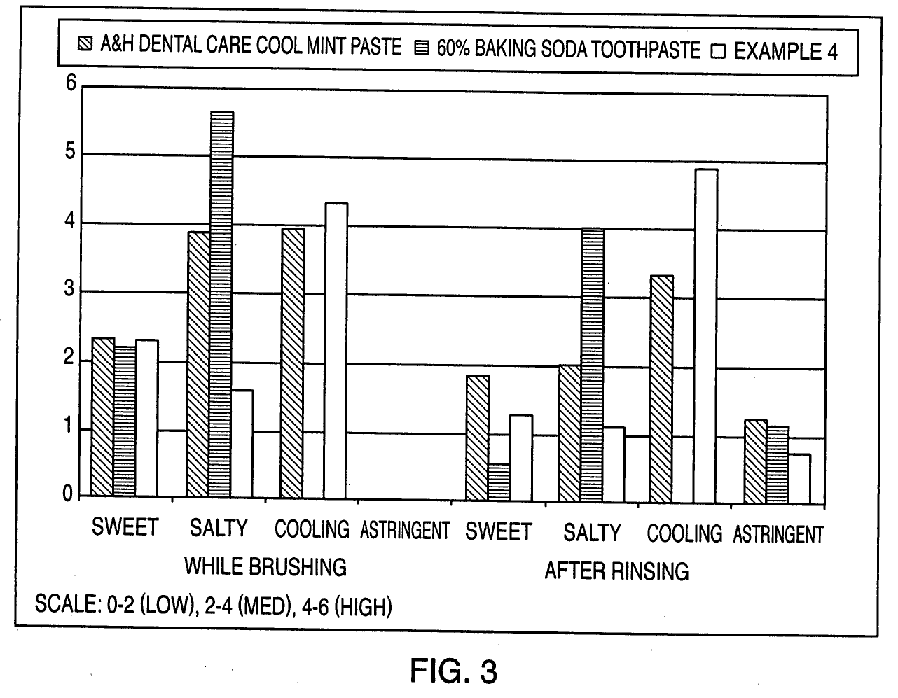

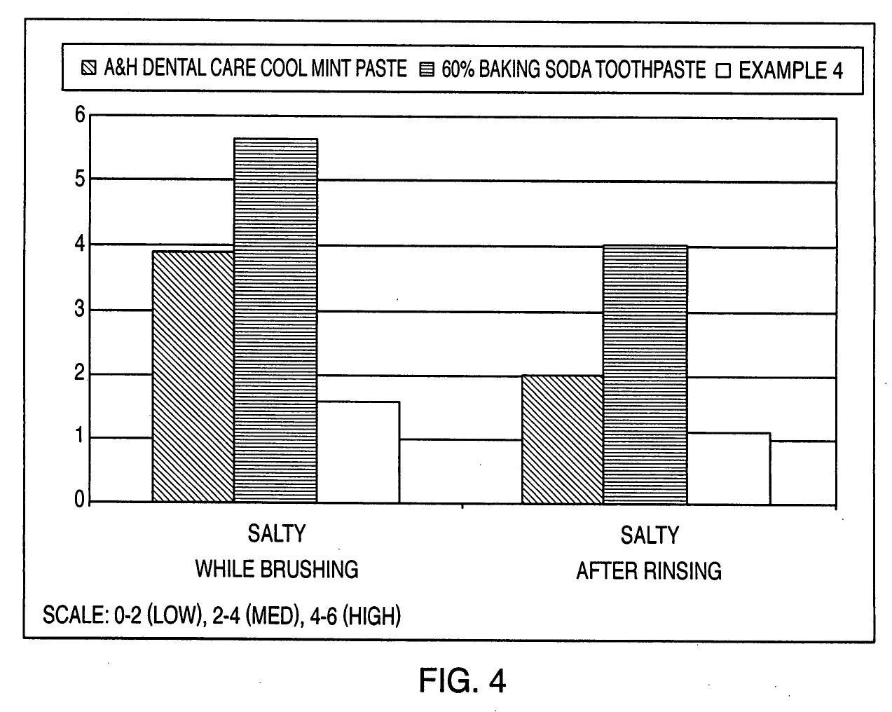

FIGS. 3 and 4 show taste testing results from six individual panelists comparing: (1) an ordinary toothpaste including 60% baking soda; (2) the toothpaste of the invention according to Example 4; and (3) the toothpaste currently marketed as ARM & HAMMER DENTAL CARES Cool Mint Paste. As can be seen in FIGS. 3 and 4, the salty taste ordinarily imparted by the 60% baking soda is greatly diminished, and almost entirely removed, in the toothpaste of the invention according to Example 4, both while brushing and after rinsing. Furthermore, the toothpaste of the invention according to Example 4 tastes much less salty than the ARM & HAMMER DENTAL CARED Cool Mint Paste. In addition, FIG. 3 shows that the toothpaste of the invention according to Example 4 imparts a cooling taste which is not present in the ordinary toothpaste including 60% baking soda, and to a higher degree than is present in the ARM & HAMMER DENTAL CARE® Cool Mint Paste.

### Examples 6-8

In Examples 6-8, denture adhesive compositions were prepared using techniques well-known to those of ordinary skill in the art.

In Example 6, the denture adhesive composition was prepared according to Example VII of U.S. Pat. No. 5,073,604, which is incorporated herein by reference in its entirety. Thus, the denture adhesive composition of Example 6 included a mixed partial salt containing zinc or strontium salts. See U.S. Pat. No. 5,073,604, col. 10, lines 6 to 25.

In Example 7, the denture adhesive composition of Example 6 was again prepared according to Example VII of U.S. Pat. No. 5,073,604, except the denture adhesive composition of Example 7 included some additional ingredients. These additional ingredients were added to the denture adhesive composition prepared according to Example VII of U.S. Pat. No. 5,073,604, and the combination was mixed until homogenous. Specifically, the denture adhesive composition of Example 7 further included the following ingredients: sodium citrate (1.0% by weight of the final denture adhesive composition), saccharin (0.05% by weight of the final denture adhesive composition), and mono-ammonium glycyrrhizinate (0.3% by weight of the final denture adhesive composition). Thus, while the denture adhesive composition of Example 7 included a mixed partial salt containing zinc or strontium salts, it also included the combination of sodium citrate, saccharin, and mono-ammonium glycyrrhizinate.

In Example 8, the denture adhesive composition of Example 6 was again prepared according to Example VII of U.S. Pat. No. 5,073,604, except the denture adhesive composition of Example 8 included some additional ingredients. These additional ingredients were added to the denture adhesive composition prepared according to Example VII of U.S. Pat. No. 5,073,604, and the combination was mixed until homogenous. Specifically, the denture adhesive composition of Example 8 further included the following ingredients: sodium citrate (1.0% by weight of the final denture adhesive composition), saccharin (0.05% by weight of the final denture adhesive composition), mono-ammonium glycyrrhizinate (0.3% by weight of the final denture adhesive composition), and the aforementioned flavor agent (0.1% by weight of the final denture adhesive composition). Thus, while the denture adhesive composition of Example 8 included a mixed partial salt containing zinc or strontium salts, it also included the combination of sodium citrate, saccharin, and mono-ammonium glycyrrhizinate, as well as the aforementioned flavor agent.

Taste testing of the denture adhesive compositions prepared according to Examples 6-8 above was conducted by a six-member panel. Specifically, the taste testing included applying an approximately 0.4 gram sample of one of the examples to the tongue, waiting approximately 30 seconds, and then evaluating the resulting taste.

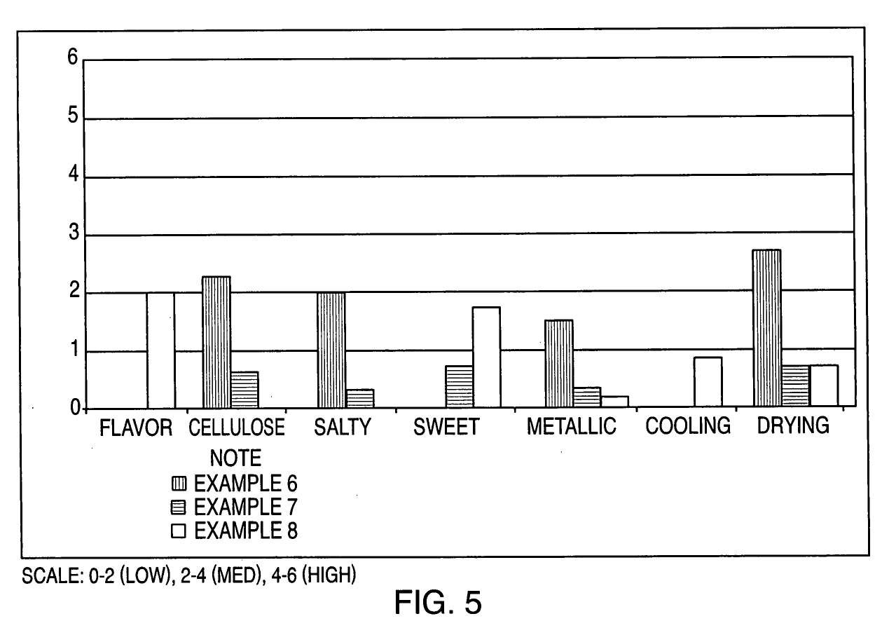

FIG. 5 shows taste testing results from six individual panelists comparing the denture adhesive compositions of Examples 6-8 described above. As can be seen in FIG. 5, the salty taste ordinarily imparted by the zinc or strontium salts present in the denture adhesive composition is greatly diminished, and almost entirely removed, in the denture adhesive compositions of the invention according to Examples 7 and 8. In addition, FIG. 5 shows that the denture adhesive compositions of the invention according to Examples 7 and 8 greatly diminished, and almost entirely removed, the metallic taste which is present in the denture adhesive composition of Example 6.

As demonstrated by the test results depicted in FIGS. 1 to 5, the salty taste of a dentifrice ordinarily imparted by a salt contained therein (e.g., sodium bicarbonate, a zinc salt, a strontium salt) is masked by the combination of the sodium citrate, the saccharin, and the mono-ammonium glycyrrhizinate.

What is claimed is:

1. An oral composition comprising a salt, an ion-channel competitor, an initial sweetener, and a delayed sweetener, wherein the ion-channel competitor, initial sweetener, and delayed sweetener are present in amounts which are effective to abate or eliminate the salty taste ordinarily imparted by the salt.

2. The oral composition of claim 1, wherein the ion-channel competitor is selected from the group consisting of sodium salts of citric acid, sodium salts of phosphoric acid, sodium salts of hydroxy acids, and calcium salts of hydroxy acids.

3. The oral composition of claim 1, wherein the ion-channel competitor includes a salt of a hydroxy acid, wherein the hydroxy acid is selected from the group consisting of glycolic acid, lactic acid, hydroxybutyric acid, mandeliec acid, glycergic acid, malic acid, tartaric acid, and mesotartaric acid.

4. The oral composition of claim 1, wherein the ion-channel competitor includes sodium citrate.

5. The oral composition of claim 4, wherein the sodium citrate is present in an amount of from about 0.25% to about 2.0% of the total weight of the oral composition.

6. The oral composition of claim 5, wherein the sodium citrate is present in an amount of about 1.0% of the total weight of the oral composition.

7. The oral composition of claim 1, wherein the initial sweetener is selected from the group consisting of saccharin, sucralose, neotame, alitame, aspartame, cyclamate, thaumatin, a dihydrochalcone, and an acesulfame potassium compound.

8. The oral composition of claim 7, wherein the initial sweetener includes saccharin.

9. The oral composition of claim 8, wherein the saccharin is present in an amount of from about 0.10% to about 0.80% of the total weight of the oral composition.

10. The oral composition of claim 9, wherein the saccharin is present in an amount of about 0.60% of the total weight of the oral composition.

11. The oral composition of claim 1, wherein the delayed sweetener includes mono-ammonium glycyrrhizinate.

12. The oral composition of claim 11, wherein the mono-ammonium glycyrrhizinate is present in an amount of from about 0.05% to about 0.50% of the total weight of the oral composition.

13. The oral composition of claim 12, wherein the mono-ammonium glycyrrhizinate is present in an amount of about 0.30% of the total weight of the oral composition.

14. The oral composition of claim 1, wherein the salt is selected from the group consisting of metallic salts of chlorines, metallic salts of fluorines, metallic salts of phosphates, metallic salts of carbonates, zinc salts, strontium salts, stannous salts, and combinations thereof.

15. The oral composition of claim 1, wherein the salt includes a zinc salt or a strontium salt.

16. An oral composition comprising a salt, an effective amount of sodium citrate, an effective amount of saccharin, and an effective amount of mono-ammonium glycyrrhizinate, wherein the effective amounts of sodium citrate, saccharin, and mono-ammonium glycyrrhizinate are effective to mask the salty taste ordinarily imparted by the salt.

17. The oral composition of claim 16, wherein the sodium citrate is present in an amount of from about 0.25% to about 2.0% of the total weight of the oral composition, the saccharin is present in an amount of from about 0.10% to about 0.80% of the total weight of the oral composition, and the mono-ammonium glycyrrhizinate is present in an amount of from about 0.05% to about 0.50% of the total weight of the oral composition.

18. The oral composition of claim 17, wherein the sodium citrate is present in an amount of about 1.0% of the total weight of the oral composition, the saccharin is present in an amount of about 0.60% of the total weight of the oral composition, and the mono-ammonium glycyrrhizinate is present in an amount of about 0.30% of the total weight of the oral composition.

19. The oral composition of claim 16, wherein the salt is selected from the group consisting of metallic salts of chlorines, metallic salts of fluorines, metallic salts of phosphates, metallic salts of carbonates, zinc salts, strontium salts, stannous salts, and combinations thereof.

20. The oral composition of claim 16, wherein the salt includes a zinc salt or a strontium salt.

21. A method of masking the salty taste of a salt in an oral composition comprising the step of adding to the oral composition an effective amount of an ion-channel competitor, an effective amount of an initial sweetener, and an effective amount of a delayed sweetener.

22. The method of claim 21, wherein the ion-channel competitor comprises sodium citrate, the initial sweetener comprises saccharin, and the delayed sweetener comprises mono-ammonium glycyrrhizinate.

23. The method of claim 22, wherein the sodium citrate is present in an amount of from about 0.25% to about 2.0% of the total weight of the oral composition, the saccharin is present in an amount of from about 0.10% to about 0.80% of the total weight of the oral composition, and the mono-ammonium glycyrrhizinate is present in an amount of from about 0.05% to about 0.50% of the total weight of the oral composition.

24. The method of claim 23, wherein the sodium citrate is present in an amount of about 1.0% of the total weight of the oral composition, the saccharin is present in an amount of about 0.60% of the total weight of the oral composition, and the mono-ammonium glycyrrhizinate is present in an amount of about 0.30% of the total weight of the oral composition.

25. The method of claim 21, wherein the salt is selected from the group consisting of metallic salts of chlorines, metallic salts of fluorines, metallic salts of phosphates, metallic salts of carbonates, zinc salts, strontium salts, stannous salts, and combinations thereof.

26. The method of claim 21, wherein the salt includes a zinc salt or a strontium salt.
